(19) United States
(12) Patent Application Publication
(10) Pub. No.: US 2026/0363972 A1
(43) Pub. Date: Nov. 19, 2026

(54) STABILITY OF SELENIUM SULFIDE DISPERSIONS

(71) Applicant: THE PROCTER & GAMBLE COMPANY, Cincinnati, OH (US)

(72) Inventors: James Anthony STAUDIGEL, Loveland, OH (US); Robert John ELSBROCK, Cincinnati, OH (US); Whitney Renee GROME, Lawrenceburg, IN (US)

(21) Appl. No.: 19/677,306

(22) Filed: May 14, 2026

Related U.S. Application Data
(60) Provisional application No. 63/805,447, filed on May 14, 2025.

(51) Int. Cl. A61K 8/23; A61K 8/22; A61K 8/368; A61K 8/81; A61Q 5/00

(52) U.S. Cl. CPC A61K 8/23; A61K 8/22; A61K 8/368; A61K 8/8147; A61K 8/8188; A61Q 5/006

(57) ABSTRACT

A composition comprising from about 40% to about 60% of selenium sulfide; from about 1% to about 3% of a first anionic polymer; and from about 0.1% to about 0.5% of a second anionic polymer wherein the anionic polymer is an acrylates copolymer.

### FIELD OF THE INVENTION

The present disclosure relates to stabilizing a selenium sulfide dispersion composition with an anionic polymer in the presence of an acrylate copolymer.

### BACKGROUND OF THE INVENTION

Selenium sulfide is a very effective anti-dandruff active that presents safe handling challenges. Dust inhalation and the resulting negative acute and chronic human health effects must be avoided. One way to mitigate handling concerns is to create a high concentration dispersion that may be safely handled in a typical personal care manufacturing environment. High concentration aqueous selenium sulfide dispersions are used as raw materials in the manufacturing of personal care compositions. Typical selenium sulfide weight content of such concentrated dispersions range between about 25% and about 50% whereas personal care compositions typically contain less than or equal to about 2% selenium sulfide.

Concentrated selenium sulfide dispersions may be prepared in central locations and shipped in various manufacturing locations. High concentrations of stable selenium sulfide are desired both for economic reasons, such as higher dispersion manufacturing productivity and lower transportation costs, as well as final product formulation flexibility. As with many dispersions, settling of the particles over time during transportation and storage is commonly encountered, requiring remixing or re-dispersing of the particles shortly before use in the final product manufacturing process. The requirement of this remixing step necessitates the use of smaller transportation packages, which may be mixed via an impeller more readily and economically. In some cases it also necessitates the frequent opening of the packages, which in turn could result in possible contamination via physical or microbial contamination and depletion of the contained preservative. Thus, improvements in dispersion and settling stability of the concentrated selenium sulfide dispersion achieve multiple benefits both in cost and quality.

The objective of the present disclosure is development of an up to 12-month stable and up to 24-month stable concentrated selenium sulfide dispersion for improved cost, quality, and supply chain flexibility. An example of a prior selenium sulfide dispersion is limited to a 21-day shelf life. The present composition contains both a surfactant (acting as both a wetting and dispersing agent) and an anionic rheology modifier. The addition of a single surfactant that acts as both a wetting agent and anionic dispersant and/or polymeric dispersant along with a small amount of rheology modifier significantly improves remix ability compared to the previous composition with minimal increase in the raw material cost. A quality dispersion with minimized particulate packing reduces the incidence of selenium sulfide particle agglomeration that translates to an elevated risk of the dense selenium particle settling in shampoo, resulting in inhomogeneity and a potential reduction in anti-dandruff benefits to the consumer.

Therefore, there is a need for a stable selenium sulfide dispersion that has up to a 12-month shelf life with improved remix, and further a need for a stable selenium sulfide dispersion that has up to a 24-month shelf life with improved remix.

### SUMMARY OF THE INVENTION

A composition comprising from about 40% to about 60% of selenium sulfide; from about 1% to about 3% of a first anionic polymer; and from about 0.1% to about 0.5% of a second anionic polymer wherein the anionic polymer is an acrylates copolymer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

While the specification concludes with claims particularly pointing out and distinctly claiming the subject matter of the present disclosure, it is believed that the composition can be more readily understood from the following description taken in connection with the accompanying drawings, in which:

FIG. 1 is a photograph of selenium sulfide 29.41% dispersion with sodium lauryl sulfate and an oxidizing agent (hydrogen peroxide) corresponding to Comparative Example 1;

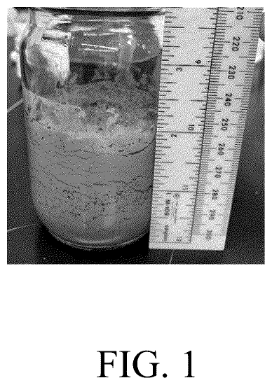

FIG. 2 is a photograph of selenium sulfide 50% dispersion with an oxidizing agent (hydrogen peroxide) corresponding to Composition Example 2;

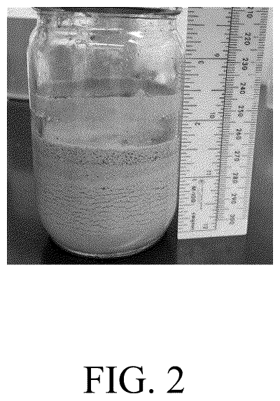

FIG. 3 is a photograph of selenium sulfide 50% dispersion with sodium polynaphthalene sulfonate and 0.06% acrylate copolymer (Rheocare® TTA) and an oxidizing agent (hydrogen peroxide) corresponding to Composition Example 3;

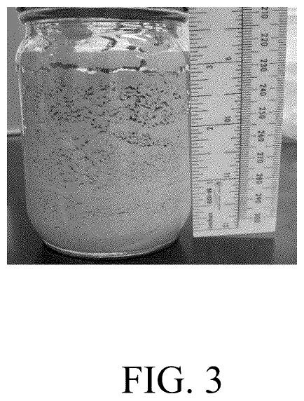

FIG. 4 is a photograph of selenium sulfide 50% dispersion with sodium polynaphthalene sulfonate and 0.15% acrylate copolymer (Rheocare® TTA) and an oxidizing agent (hydrogen peroxide) corresponding to Composition Example 4;

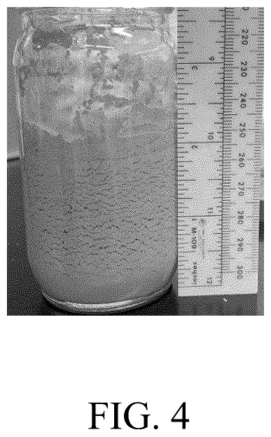

FIG. 5 is a photograph of selenium sulfide 50% dispersion with sodium polynaphthalene sulfonate and 0.30% acrylate copolymer (Rheocare® TTA) and an oxidizing agent (hydrogen peroxide) corresponding to Composition Example 5;

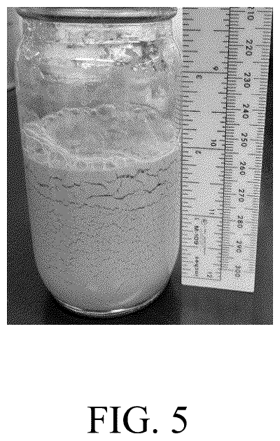

FIG. 6 is a photograph of selenium sulfide 50% dispersion with sodium polynaphthalene sulfonate and 0.60% acrylate copolymer (Rheocare® TTA) and an oxidizing agent (hydrogen peroxide) corresponding to Composition Example 6;

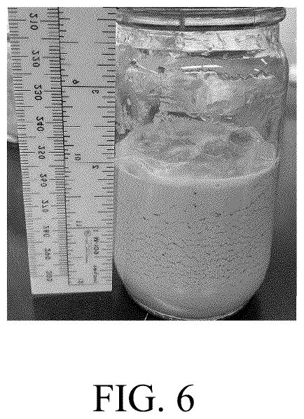

FIG. 7 is a photograph of selenium sulfide 50% dispersion with sodium lauryl sulfate and no oxidizing agent (hydrogen peroxide) corresponding to Comparative Example 7;

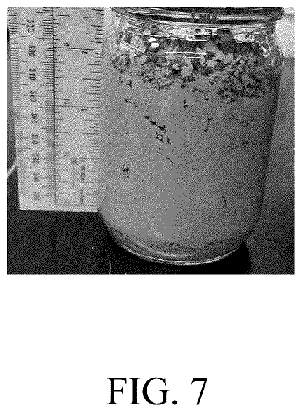

FIG. 8 is a photograph of selenium sulfide 50% dispersion with sodium polynaphthalene sulfonate and no oxidizing agent (hydrogen peroxide) corresponding to Composition Example 8;

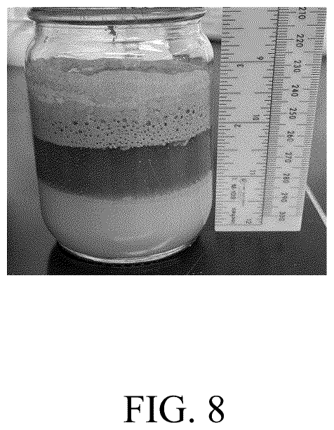

FIG. 9 is a photograph of selenium sulfide 50% dispersion with sodium polynaphthalene sulfonate and 0.06% acrylate copolymer (Rheocare® TTA) and no oxidizing agent (hydrogen peroxide) corresponding to Composition Example 9;

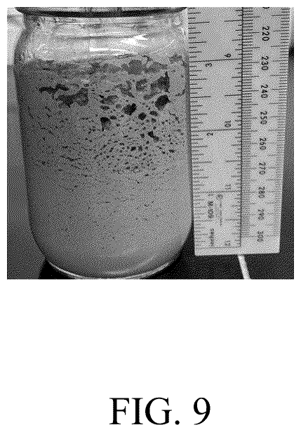

FIG. 10 is a photograph of selenium sulfide 50% dispersion with sodium polynaphthalene sulfonate and 0.15% acrylate copolymer (Rheocare® TTA) and no oxidizing agent (hydrogen peroxide) corresponding to Composition Example 10;

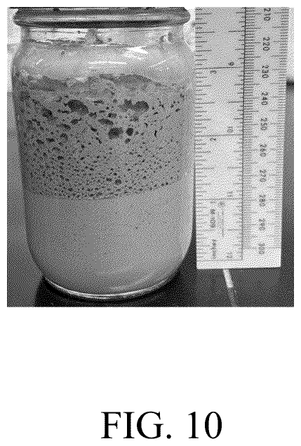

FIG. 11 is a photograph of selenium sulfide 50% dispersion with sodium polynaphthalene sulfonate and 0.30% acrylate copolymer (Rheocare® TTA) and no oxidizing agent (hydrogen peroxide) corresponding to Composition Example 11;

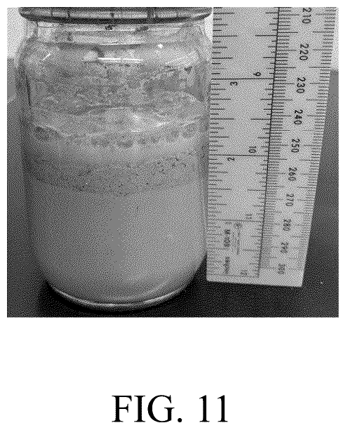

FIG. 12 is a photograph of selenium sulfide 50% dispersion with sodium polynaphthalene sulfonate and 0.60% acrylate copolymer (Rheocare® TTA) and no oxidizing agent (hydrogen peroxide) corresponding to Composition Example 12.

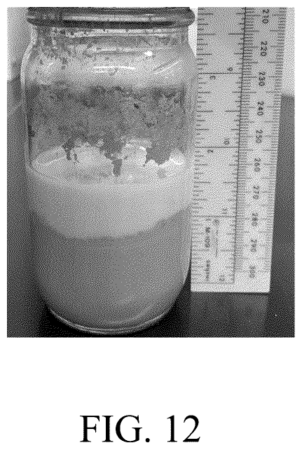

FIG. 13 is a depiction of a penetration method.

FIG. 14 is a graph depicting maximum penetration force comparing 0% acrylate copolymer vs. 0.20% acrylate copolymer across various time intervals and temperatures.

FIG. 15 is a graph depicting maximum penetration force comparing 0% acrylate copolymer vs. 0.60% acrylate copolymer across various time intervals and temperatures.

### DETAILED DESCRIPTION OF THE INVENTION

While the specification concludes with claims particularly pointing out and distinctly claiming the invention, it is believed that the present disclosure will be better understood from the following description.

As used herein, the articles including “a” and “an” when used in a claim, are understood to mean one or more of what is claimed or described.

As used herein, “comprising” means that other steps and other ingredients which do not affect the end result can be added. This term encompasses the terms “consisting of” and “consisting essentially of.”

As used herein, the term “fluid” includes liquids, gels, emulsions, or suspensions.

As used herein, “mixtures” is meant to include a simple combination of materials and any compounds that may result from their combination.

As used herein, “molecular weight” or “M.Wt.” refers to the weight average molecular weight unless otherwise stated. Molecular weight is measured using industry standard method, gel permeation chromatography (“GPC”).

As used herein, “personal care composition” includes hair care products such as shampoos, conditioners, conditioning shampoos, hair colorants, as well as shower gels, liquid hand cleansers, facial cleansers, laundry detergent, dish detergent, and other surfactant-based liquid compositions.

As used herein, “dispersion” is defined as a mixture where one material is spread evenly throughout another, creating a system with a continuous phase and dispersed particles.

As used herein, “cake” is defined as the physical settling of dispersed particles that must be remixed in order to provide homogeneity of the composition.

As used herein, “substantially free” means less than 3%, less than 2%, less than 1%, less than 0.5%, less than 0.25%, less than 0.1%, less than 0.05%, less than 0.01%, less than 0.001%, and/or free of. As used herein, “free of” means 0%.

As used herein, the terms “include,” “includes,” and “including,” are meant to be non-limiting and are understood to mean “comprise,” “comprises,” and “comprising,” respectively.

All percentages, parts and ratios are based upon the total weight of the compositions of the present disclosure, unless otherwise specified. All such weights as they pertain to listed ingredients are based on the active level and, therefore, do not include carriers or by-products that may be included in commercially available materials.

Unless otherwise noted, all component or composition levels are in reference to the active portion of that component or composition, and are exclusive of impurities, for example, residual solvents or by-products, which may be present in commercially available sources of such components or compositions.

The test methods disclosed in the Test Methods Section of the present application can be used to determine the respective values of the parameters of Applicants' inventions.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Where amount ranges are given, these are to be understood as being the total amount of said ingredient in the composition, or where more than one species fall within the scope of the ingredient definition, the total amount of all ingredients fitting that definition, in the composition. For example, if the composition comprises from 1% to 5% fatty alcohol, then a composition comprising 2% stearyl alcohol and 1% cetyl alcohol and no other fatty alcohol, would fall within this scope.

The amount of each particular ingredient or mixtures thereof described hereinafter can account for up to 100% (or 100%) of the total amount of the ingredient(s) in the shampoo composition.

The present disclosure is directed to development of a composition that may be used to make the manufacturing of personal care products more safe and cost effective by utilizing bulk quantities of stable selenium sulfide composition. The previous composition dispersion settles into a hard packed cake over time resulting in a very short shelf life of 21 days or 3 weeks. This provides several challenges including remixing, transportation, and contamination due to extra handling.

The present disclosure is directed to a dispersion composition containing, in addition to selenium sulfide, sodium polynaphthalene sulfonate, and preservative, and an acrylates copolymer (as a rheology modifier). This rheology modifier significantly improves settling stability by increasing the low shear viscosity of the composition and more importantly, it creates a swollen microgel that prevents the formation of a hard packed selenium sulfide cake over time. Thus, it extends the usable shelf life, enables the transportation of the dispersion in significantly larger containers, and it reduces the time and energy required for remixing before use. This acrylate copolymer is commonly used as a rheology modifier in currently marketed anti-dandruff shampoos; thus, the risk of lack of compatibility of the concentrated selenium sulfide dispersion with the final shampoo compositions is avoided.

### Selenium Sulfide

The formation of three eight-membered selenium sulfide ring molecules in the molten mixtures of sulfur and selenium is well established. Selenium sulfide is generally regarded as a compound having one mole of selenium and two moles of sulfur. However, it may take the form of a cyclic structure, SexSy, wherein x+y=8. Selenium sulfide is a difficult material to work with because it is very dense, approximately 3.0 g/cm3. It presents an inhalation hazard and given its chemical composition, is very hydrophobic which makes it incompatible with water. Therefore, in an aqueous dispersion composition there must be a surfactant or other wetting agent present. The selenium sulfide has a particle size D100 of less than 30 microns, D90 of less than 20 microns, and a D50 of between 4 microns and 12 microns. These measurements are made using a forward laser light scattering device (e.g., a Malvern 3600 instrument). Selenium sulfide is present in the compositions of this invention at a level of from about 40% to about 60%, from about 45% to about 60%, from about 50% to about 60%.

### Anionic Polymer

Anionic polymers may be used in the composition. A nonlimiting example of such an anionic polymer is sodium polynaphthalene sulfonate. Sodium polynaphthalene sulfonate, for example, may have a molecular weight of from 8000 g/mol to 30,000 g/mol, from 10000 g/mol to 20,000 g/mol, from 12,000 g/mol to 15,000 g/mol.

Anionic polymers useful herein include, for example, sodium polynaphthalene sulfonate, Sodium Lignosulfonate, sodium carboxymethyl modified maleic anhydride cellulose, Sodium salt of hydrophobically copolymer, Sodium polyacrylate, sodium polymethacrylate, ammonium polyacrylate, ammonium polymethacrylate, Sodium salt of polymethacrylic acid, sodium polynaphthalene sulfonate, and sodium carboxymethyl cellulose, sodium polynaphthalene sulfonate, sodium polynaphthalene sulfonate having a tradename Darvan1 Spray Dried, supplied from Vanderbilt Minerals having a molecular weight of 3,000 g/mol in comparison to standards of sodium poly(styrenesulfonate) and a charge density of from 3.5 to 4.0 meq/g.

The composition may have from about 1% to about 3% of an anionic polymer, from about 1.5% to about 2.5%, and from about 1.5% to about 2%.

### Stabilizing Polymers

The composition may comprise a stabilizing polymer to increase the viscosity of the composition and/or create a structured microgel network in the composition. Surfactant-activated microgels are microgels whose properties, particularly their swelling and interaction with other microgels, are influenced by the presence of surfactants. These microgels are typically composed of hydrophobic and hydrophilic polymers, and the surfactants act as mediators, enhancing the interaction and dispersion of the microgels within a solution. Suitable stabilizing polymers can be used. The selenium dispersion composition may comprise from about 0.1% to about 2.0% of a stabilizing polymer, from about 0.1% to about 1.0% of a stabilizing polymer, and from about 0.1% to about 0.5% of a stabilizing polymer, from about 0.1% to about 0.3%, and from about 0.1% to about 0.2%. The stabilizing polymer may be a polyacrylate, polyacrylamide stabilizer. The stabilizing polymer may be an anionic stabilizing polymer that are homopolymers based on acrylic acid, methacrylic acid or other related derivatives, non-limiting examples include polyacrylate, polymethacrylate, polyethylacrylate, and polyacrylamide.

The stabilizing polymers may be alkali swellable and hydrophobically-modified alkali swellable acrylic copolymers or methacrylate copolymers, non-limiting examples include acrylic acid/acrylonitrogens copolymer, acrylates/steareth-20 itaconate copolymer, acrylates/ceteth-20 itaconate copolymer, Acrylates/Aminoacrylates/C10-30 Alkyl PEG-20 Itaconate Copolymer, acrylates/aminoacrylates copolymer, acrylates/steareth-20 methacrylate copolymer, acrylates/beheneth-25 methacrylate copolymer, acrylates/steareth-20 methacrylate crosspolymer, acrylates/beheneth-25 methacrylate/HEMA crosspolymer, acrylates/vinyl neodecanoate crosspolymer, acrylates/vinyl isodecanoate crosspolymer, Acrylates/Palmeth-25 Acrylate Copolymer, Acrylic Acid/Acrylamidomethyl Propane Sulfonic Acid Copolymer, and acrylates/C10-C30 alkyl acrylate crosspolymer.

The stabilizing polymers may be soluble crosslinked acrylic polymers, a non-limiting example includes carbomers.

The stabilizing polymers may be associative polymeric thickeners, non-limiting examples include: hydrophobically modified, alkali swellable emulsions, non-limiting examples include hydrophobically modified polypolyacrylates; hydrophobically modified polyacrylic acids, and hydrophobically modified polyacrylamides; hydrophobically modified polyethers wherein these materials may have a hydrophobe that can be selected from cetyl, stearyl, oleayl, and combinations thereof.

The stabilizing polymers may be used in combination with polyvinylpyrrolidone, crosslinked polyvinylpyrrolidone and derivatives. The stabilizing polymers may be combined with polyvinyalcohol and derivatives. The stabilizing polymers may be combined with polyethyleneimine and derivatives.

The stabilizing polymers may be combined with alginic acid based materials, non-limiting examples include sodium alginate, and alginic acid propylene glycol esters.

The stabilizing polymers may be used in combination with polyurethane polymers, non-limiting examples include: hydrophobically modified alkoxylated urethane polymers, non-limiting examples include PEG-150/decyl alcohol/SMDI copolymer, PEG-150/stearyl alcohol/SMDI copolymer, polyurethane-39.

The stabilizing polymers may be combined with an associative polymeric thickeners, non-limiting examples include: hydrophobically modified cellulose derivatives; and a hydrophilic portion of repeating ethylene oxide groups with repeat units from about 10 to about 300, from about 30 to about 200, from about 40 to about 150. Non-limiting examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate.

The stabilizing polymers may be combined with cellulose and derivatives, non-limiting examples include microcrystalline cellulose, carboxymethylcelluloses, hydroxyethylcellulose, hydroxypropylcellulose, hydroxypropyl methylcellulose, methylcellulose, ethyl cellulose; nitro cellulose; cellulose sulfate; cellulose powder; hydrophobically modified celluloses.

The stabilizing polymers may be combined with a guar and guar derivatives, non-limiting examples include hydroxypropyl guar, and hydroxypropyl guar hydroxypropyl trimonium chloride.

The stabilizing polymers may be combined with polyethylene oxide; polypropylene oxide; and POE-PPO copolymers.

The stabilizing polymers may be combined with polyalkylene glycols characterized by the general formula:

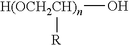

wherein R is hydrogen, methyl, or mixtures thereof, and further hydrogen, and n is an integer having an average from 2,000-180,000, or from 7,000-90,000, or from 7,000-45,000. Non-limiting examples of this class include PEG-7M, PEG-14M, PEG-23M, PEG-25M, PEG-45M, PEG-90M, or PEG-100M.

The stabilizing polymers may be combined with silicas, non-limiting examples include fumed silica, precipitated silica, and silicone-surface treated silica.

The stabilizing polymers may be combined with water-swellable clays, non-limiting examples include laponite, bentolite, montmorilonite, smectite, and hectonite.

The stabilizing polymers may be combined with gums, non-limiting examples include xanthan gum, guar gum, hydroxypropyl guar gum, Arabia gum, tragacanth, galactan, carob gum, karaya gum, and locust bean gum.

The stabilizing polymers may be combined with, dibenzylidene sorbitol, karaggenan, pectin, agar, quince seed (Cydonia oblonga Mill), starch (from rice, corn, potato, wheat, etc), starch-derivatives (e.g. carboxymethyl starch, methylhydroxypropyl starch), algae extracts, dextran, succinoglucan, and pulleran,

Non-limiting examples of stabilizing polymers include acrylamide/ammonium acrylate copolymer (and) polyisobutene (and) polysorbate 20; acrylamide/sodium acryloyldimethyl taurate copolymer/isohexadecane/polysorbate 80, ammonium acryloyldimethyltaurate/VP copolymer, Sodium Acrylate/Sodium Acryloyldimethyl Taurate Copolymer, acrylates copolymer, Acrylates Crosspolymer-4, Acrylates Crosspolymer-3, acrylates/beheneth-25 methacrylate copolymer, acrylates/C10-C30 alkyl acrylate crosspolymer, acrylates/steareth-20 itaconate copolymer, ammonium polyacrylate/Isohexadecane/PEG-40 castor oil; carbomer, sodium carbomer, polyvinylpyrrolidone (PVP), polyacrylamide/C13-14 isoparaffin/laureth-7, crosslinked polyacrylate 13/polyisobutene/polysorbate 20, polyacrylate crosspolymer-6, polyamide-3, polyquaternium-37 (and) hydrogenated polydecene (and) trideceth-6, Acrylamide/Sodium Acryloyldimethyltaurate/Acrylic Acid Copolymer, sodium acrylate/acryloyldimethyltaurate/dimethylacrylamide, crosspolymer (and) isohexadecane (and) polysorbate 60, sodium polyacrylate. Exemplary commercially-available stabilizing polymers include ACULYN™ 28, ACULYN™ 33, ACULYN™ 88, ACULYN™ 22, ACULYN™ Excel, Carbopol® Aqua SF-1, Carbopol® ETD 2020, Carbopol® Ultrez 20, Carbopol® Ultrez 21, Carbopol® Ultrez 10, Carbopol® Ultrez 30, Carbopol® 1342, Carbopol® Aqua SF-2 Polymer, Sepigel™ 305, Simulgel™ 600, Sepimax Zen, Carbopol® SMART 1000, Rheocare® TTA, Rheomer® SC-Plus, STRUCTURE® PLUS, Aristoflex® AVC, Stabylen 30 and combinations thereof.

Sodium polynaphthalene sulfonate may have a nonlimiting tradename Darvan1 Spray Dried, supplied from RT Vanderbilt having a molecular weight of about 3,000 g/mol in comparison to standards of sodium poly(styrenesulfonate) and a charge density of from about 3.5 to about 4.0 meq/g.

### Dispersants

The composition may contain a dispersant. Typically, small particle dispersions require stabilization to prevent particle aggregation/agglomeration and also stabilization to prevent particle settling, The former is achieved by the presence of one or more surfactants or one or more polymeric dispersants which protect the particles sterically and/or electrostatically, The latter is achieved by the presence of a rheology modifier, which increases the viscosity especially in the low shear range. Sterle and/or electrostatic particle protection can be achieved by the addition in the dispersion compositions of anionic, non-ionic, cationic, amphoteric, zwitterionic surfactants or polymeric dispersants. Such surfactants can thereby act as dispersants, including anionic surfactants. Typically, polymeric dispersant molecules comprise of functional groups that anchor on the particle and functional groups that are compatible with the carrier of the dispersion Anchoring group is a selected from non-limiting examples such as carboxyls, carboxylic acid esters, hydroxyls, sulfonates, sulfates, phosphates, phosphonates, nitros, carbohydrates, ammonium salts, phosphate esters, carbonyls, aminos, amides, imides, aliphatic hydrocarbons, aromatic hydrocarbons, heterocydic groups, polypropyleneoxides, silicones, fluorocarbons, polyesters, urethanes and mixtures thereof.

The stabilizing group is selected from non-limiting examples such as polyethyleneoxide, polyethylene glycols, polypropylene glycol, polyethylene glycol alkyl, alkyl glycols, 30 alkyl glycol ether, polyethylene glycol esters, polyalkylene oxide, polypropyleneoxides, polyglycerides, carboxyl, carboxylic acid esters, hydroxyl, sulfonate, sulfate, phosphate phosphonate, nitro, carbohydrate, ammonium salts, phosphate esters, carbonyl, amino, amide, imide, aliphatic hydrocarbons, aromatic hydrocarbons, heterocyclic groups, polypropyleneoxides, silicones, fluorocarbons, polyesters and mixtures thereof. Sodium polynaphthalene sulfonate may also be considered as a dispersant.

### Preservative

The composition may comprise a preservative. The preservative may be sodium salicylate, sodium benzoate. These preservatives may be present from about 0.1% to about 1.0%, from about 0.5% to about 0.75%, from about 0.25% to about 0.5%. The composition may comprise the preservatives Methylchloroisothiazolinone (CMIT) at about 4 ppm and Methylisothiazolinone (MIT) at about 1 ppm sold under the tradename Kathon™. Another preservative that may be used in the composition is phenoxyethanol up to 1% in a composition.

### Oxidizing Agent

The composition may further comprise an oxidizing agent. The oxidizing agent may be hydrogen peroxide, sulfur, colloidal sulfur or other oxidizing agent known to those skilled in the art. The oxidizing agent may be present from about 0.1% to about 1.0%, from about 0.5% to about 0.75%, from about 0.25% to about 0.5%.

### Reducing Agent

The composition may further comprise a reducing agent. The reducing agent may be hexadimethrine chloride, alpha-hydroxy acids like glycolic acid and lactic acid, and reducing sugars like glucose, galactose and xylose or other reducing agent known to those skilled in the art. Hexadimethrine chloride is a quaternary ammonium salt compound. The reducing agent may be present from about 0.1% to about 1.0%, from about 0.5% to about 0.75%, from about 0.25% to about 0.5%.

### Liquid Carrier

The composition may include a liquid carrier can facilitate an appropriate liquid viscosity and rheology. The composition may include, by weight of the composition, about 38% to about 58%, of a liquid carrier, about 45% to about 50%, and from about 47% to about 48%.

A liquid carrier can be water, or can be a miscible mixture of water and organic solvent. A liquid carrier can be water with minimal or no significant concentrations of organic solvent, except as otherwise incidentally incorporated into the composition as minor ingredients of other essential or optional components. Suitable organic solvents can include water solutions of lower alkyl alcohols and polyhydric alcohols. Useful lower alkyl alcohols include monohydric alcohols having 1 to 6 carbons, such as ethanol and isopropanol. Exemplary polyhydric alcohols include propylene glycol, dipropylene glycol, hexylene glycol, glycerin, and propane diol.

### Composition Preparation

In an 8 oz. glass jar, water, anionic polymer, and stabilizing agent are added with overhead mixing via a marine impeller. The selenium sulfide powder is then added and mixed well. Lastly, the preservative and optional oxidizing and/or reducing agents are added and mixed well.

### Test Methods

### Penetration Method

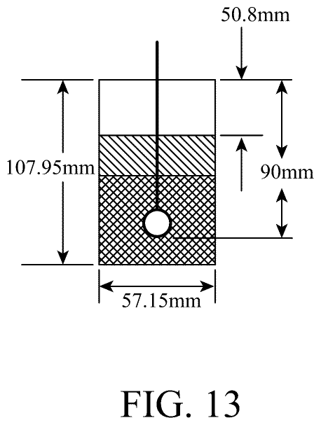

To measure the amount of caking of the selenium sulfide dispersion, a Texture Analyzer TA-XT2i (Texture Technologies Corp, NY, USA) is used with at least a 5 kg load cell and a 0.75 inch ball probe at ambient conditions, with the probe zero point at an article top surface using 0.5 gram-force to register a probe height, and a 2 gram-force to commence data collection for both force and distance. A compressive force (kg) is measured at a compression rate of 1 mm/see over a depth of 5 mm, ensuring that the personal care article or composition form a flat surface over contact area with the ball probe, near the center of the article or composition. Measurements are repeated as needed (e.g., at least 3 times) to obtain a representative average value. When using a kg load cell some samples may exceed capacity, in this case the maximum compression depth will be less than the set depth of 5 mm, specified in the procedure. FIG. 13 is a depiction of the above penetration method.

### Settling Height

The settling height is a measurement of the visible selenium sulfide cake using a ruler. This measurement occurs in the same 8 oz jar used to prepare the composition. The measurement may be taken at a) room temperature or b) an elevated temperature intended to speed up any settling that occurs over time at room temperature and may be predictive of settling over longer periods of time.

### EXAMPLES

The following are non-limiting examples of the composition described herein. The examples are prepared by conventional techniques and include adding the ingredients one by one and mixing until homogeneous or dissolved and adding heat as necessary to dissolve particular ingredients. It will be appreciated that other modifications of the composition within the skill of those in the personal care composition art can be undertaken without departing from the spirit and scope of this composition. All parts, percentages, and ratios herein are by weight unless otherwise specified. Some components may come from suppliers as dilute solutions. The amount stated reflects the weight percent of the active material, unless otherwise specified.

Dispersions containing Hydrogen Peroxide

| | Comparative | Comparative | Composition | Composition | Composition | Composition |
|---|---|---|---|---|---|---|
| | Example | Example | Example | Example | Example | Example |
| INGREDIENTS (%) | 1 | 2 | 3 | 4 | 5 | 6 |
| Selenium Sulfide ¹ | 29.41 | 50.0% | 50.0 | 50.0 | 50.0 | 50.0 |
| Sodium | — | 1.50 | 1.5 | 1.5 | 1.5 | 1.5 |
| polynaphthalene | | | | | | |
| sulfonate ² | | | | | | |
| Sodium Lauryl | 1.23 | — | — | — | — | — |
| Sulfate ³ | | | | | | |
| Acrylates copolymer ⁴ | — | — | 0.06 | 0.15 | 0.30 | 0.60 |
| Hydrogen Peroxide ⁵ | 0.37 | 0.25 | 0.25 | 0.25 | 0.25 | 0.25 |
| Sodium Benzoate ⁶ | — | 0.25 | 0.25 | 0.25 | 0.25 | 0.25 |
| Sodium Salicylate ⁷ | — | 0.25 | 0.25 | 0.25 | 0.25 | 0.25 |
| Water | QS | QS | QS | QS | QS | QS |

All ingredient percentages listed above are in percent active material.
¹ Selenium Sulfide, from Eskay Specialty Chemicals
² Darvan 1, from R T Vanderbilt
³ Sodium Lauryl Sulfate 29%, from the Procter & Gamble Company
⁴ Rheocare TTA from BASF
⁵ Hydrogen Peroxide 25% Super D, from PeroxyChem LLC
⁶ Sodium Benzoate Dense NF/FCC, from Emerald Performance Materials
⁷ Sodium Salicylate, from JQC (Huayin) Pharmaceutical Co., Ltd.

Dispersions without Hydrogen Peroxide

| | Comparative | Comparative | Composition | Composition | Composition | Composition |
|---|---|---|---|---|---|---|
| | Example | Example | Example | Example | Example | Example |
| INGREDIENTS (%) | 7 | 8 | 9 | 10 | 11 | 12 |
| Selenium Sulfide ¹ | 50.0% | 50.0% | 50.0 | 50.0 | 50.0 | 50.0 |
| Sodium | — | 1.50 | 1.5 | 1.5 | 1.5 | 1.5 |
| polynaphthalene | | | | | | |
| sulfonate ² | | | | | | |
| Sodium Lauryl | 1.23 | — | — | — | — | — |
| Sulfate ³ | | | | | | |
| Acrylates copolymer ⁴ | — | — | 0.06 | 0.15 | 0.30 | 0.60 |
| Hydrogen Peroxide ⁵ | — | — | — | — | — | — |
| Sodium Benzoate ⁶ | — | 0.25 | 0.25 | 0.25 | 0.25 | 0.25 |
| Sodium Salicylate ⁷ | — | 0.25 | 0.25 | 0.25 | 0.25 | 0.25 |
| Water | QS | QS | QS | QS | QS | QS |

All ingredient percentages listed above are in percent active material.
¹ Selenium Sulfide, from Eskay Specialty Chemicals
² Darvan 1, from R T Vanderbilt
³ Sodium Lauryl Sulfate 29%, from the Procter & Gamble Company
⁴ Rheocare TTA from BASF
⁵ Hydrogen Peroxide 25% Super D, from PeroxyChem LLC
⁶ Sodium Benzoate Dense NF/FCC, from Emerald Performance Materials
⁷ Sodium Salicylate, from JQC (Huayin) Pharmaceutical Co., Ltd.

### Results

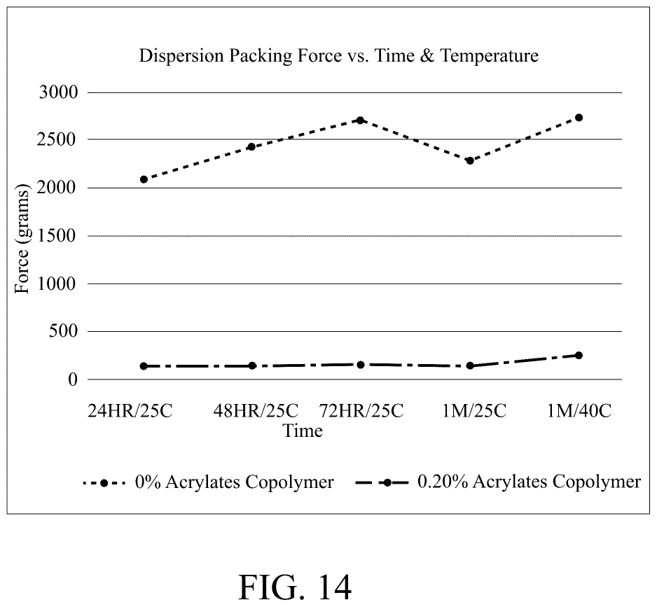

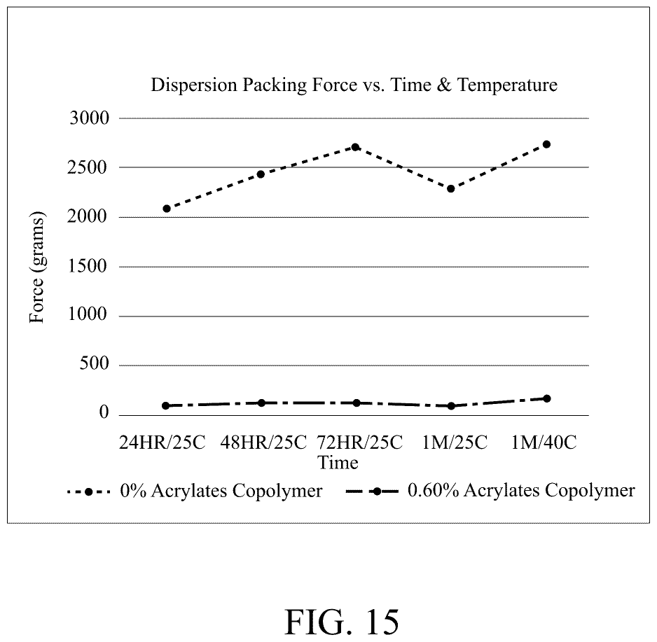

FIGS. 14 and 15 represent the degree of selenium sulfide packing based on the acrylate copolymer levels—The higher the force the greater the amount of selenium sulfide particle settling. The graphs indicate that a concentration of 0.2 and 0.6% acrylate copolymer, respectively, minimizes the settling of selenium sulfide thereby providing ease of redispersion prior to introducing into a personal care composition. An acceptable amount of settling is demonstrated at one month at 40 C storage condition which is predictive of one year ambient storage conditions. This insures homogeneity and thorough redispersion of selenium sulfide particles prior to introduction into a personal care composition.

### Dispersions Containing Hydrogen Peroxide-Timepoint 24 Hours at 40 C, No Agitation

| | Comparative | Comparative | | | | |
|---|---|---|---|---|---|---|
| | Example 1 | Example 2 | Example 3 | Example 4 | Example 5 | Example 6 |
| Cake Height | 51 | 32 | 76 | 32 | 32 | 32 |
| (in mm) | | | | | | |
| Settling | C | A | C | A | A | A |
| Stability | | | | | | |
| Rating | | | | | | |
| Rating of | 3 | 2 | 2 | 2 | 2 | 2 |
| redispersibility | | | | | | |
| of settled | | | | | | |
| particle cake. | | | | | | |

Dispersions without Hydrogen Peroxide-Timepoint 24 Hours at 40 C, No Agitation

| | Comparative | Comparative | | | | |
|---|---|---|---|---|---|---|
| | Example 7 | Example 8 | Example 9 | Example 10 | Example 11 | Example12 |
| Cake Height | 70 | 25 | 38 | 38 | 38 | 38 |
| (in mm) | | | | | | |
| Settling | C | A | A | A | A | A |
| Stability | | | | | | |
| Rating | | | | | | |
| Rating of | 3 | 3 | 2 | 2 | 2 | 3 |
| redispersibility | | | | | | |
| of settled | | | | | | |
| particle cake. | | | | | | |

### Settling Stability Rating

A: Very minimum selenium sulfide settling after 24 hours at 40 C storage condition.
B: Significant selenium sulfide settling after 24 hours at 40 C storage condition
C: Most/all selenium sulfide particles settle after 24 hours at 40 C storage condition.
   Rating of re-dispersibility of settled particle cake
1: No cake formation
2: Soft cake, easily redispersed.
3. Medium cake; cake requires mixing energy and time to redisperse.
4: Hard cake; substantially all the cake stays separated from the carrier even after stirring.

The results of the composition suggest that:
a. The least desirable results which have either the hardest cake and/or largest cake height are observed in the samples that do not contain acrylates copolymer (Ex 1, 2, 7, & 8).
b. Composition containing too much stabilizing polymer show poor results (Exp 6 & 12). Even if the dispersion does not form a hard cake, it phase separates which is not as easily re-dispersible.
c. When combined, a polymeric dispersant and stabilizing polymer result in production of a soft cake dispersion that is easily re-dispersed and minimizes cake height.

### Additional Examples/Combinations

A. A composition comprising:
a) from about 40% to about 60% of selenium sulfide;
b) from about 1% to about 3% of a first anionic polymer; and
c) from about 0.1% to about 0.5% of a second anionic polymer wherein the anionic polymer is an acrylates copolymer.
B. A composition according to Paragraph A, wherein A composition according to claim 1 wherein the selenium sulfide is from about 40% to about 60%.
C. A composition according to Paragraph A-B, wherein the selenium sulfide is from about 50% to about 60%.
D. A composition according to Paragraph A-C, wherein the anionic polymer is sodium polynaphthalene sulfonate.
E. A composition according to Paragraph A-D, wherein the sodium polynaphthalene sulfonate is from about 1% to about 2%.
F. A composition according to Paragraph A-E, wherein the acrylate copolymer is from about 0.1% to about 0.3%.
G. A composition according to Paragraph A-F, wherein the acrylate coplolymer is selected from the group consisting of 2-Propenoic acid, 2-methyl-, polymer with butyl 2-propenoate and ethyl 2-propenoate, acrylamide/ammonium acrylate copolymer (and) polyisobutene (and) polysorbate 20; acrylamide/sodium acryloyldimethyl taurate copolymer/isohexadecane/polysorbate 80, ammonium acryloyldimethyltaurate/VP copolymer, Sodium Acrylate/Sodium Acryloyldimethyl Taurate Copolymer, acrylates copolymer, Acrylates Crosspolymer-4, Acrylates Crosspolymer-3, acrylates/beheneth-25 methacrylate copolymer, acrylates/C10-C30 alkyl acrylate crosspolymer, acrylates/steareth-20 itaconate copolymer, ammonium polyacrylate/Isohexadecane/PEG-40 castor oil; carbomer, sodium carbomer, crosslinked polyvinylpyrrolidone (PVP), polyacrylamide/C13-14 isoparaffin/laureth-7, polyacrylate 13/polyisobutene/polysorbate 20, polyacrylate crosspolymer-6, polyamide-3, polyquaternium-37 (and) hydrogenated polydecene (and) trideceth-6, Acrylamide/Sodium Acryloyldimethyltaurate/Acrylic Acid Copolymer, sodium acrylate/acryloyldimethyltaurate/dimethylacrylamide, crosspolymer (and) isohexadecane (and) polysorbate 60, sodium polyacrylate.
H. A composition according to paragraph A-G, wherein the composition has a selenium sulfide cake 90 mm penetration depth peak force of less than 200 grams after 24 hours at 40 C.
I. A composition according to paragraph A-H, wherein the composition has a cake height of greater than 30 mm after 24 hours at 40 C.
J. A composition according to paragraph A-I, further comprising an oxidizing agent.
K. A composition according to paragraph A-J, wherein the oxidizing agent is from about 0.1% to about 0.4% of an oxidizing agent.
L. A composition according to paragraph A-K, wherein the oxidizing agent is selected from the group consisting of hydrogen peroxide, sulfur, colloidal sulfur and mixtures thereof.
M. A composition according to paragraph A-L, further comprising a preservative.
N. A composition according to paragraph A-M, wherein the preservative is selected from the group consisting of sodium salicylate, sodium benzoate, phenoxyethanol, Methylchloroisothiazolinone (CMIT) and Methylisothiazolinone (MIT) and mixtures thereof.
O. A composition according to paragraph A-N, wherein the preservative is from about 0.1% to about 1.0%.
P. A composition according to paragraph A-O, further comprising from about 38% to about 58% of a liquid carrier.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular forms of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of the present disclosure.

What is claimed is:

1. A composition comprising:
a. from about 40% to about 60% of selenium sulfide;
b. from about 1% to about 3% of a first anionic polymer; and
c. from about 0.1% to about 0.5% of a second anionic polymer wherein the anionic polymer is an acrylates copolymer.

2. A composition according to claim 1 wherein the selenium sulfide is from about 45% to about 60%.

3. A composition according to claim 1 wherein the selenium sulfide is from about 50% to about 60%.

4. A composition according to claim 1 wherein the first anionic polymer is sodium polynaphthalene sulfonate.

5. A composition according to claim 1 wherein the sodium polynaphthalene sulfonate is from about 1% to about 2%.

6. A composition according to claim 1 wherein the acrylate copolymer is from about 0.1% to about 0.3%.

7. A composition according to claim 1 wherein the acrylate coplolymer is selected from the group consisting of 2-Propenoic acid, 2-methyl-, polymer with butyl 2-propenoate and ethyl 2-propenoate, acrylamide/ammonium acrylate copolymer (and) polyisobutene (and) polysorbate 20; acrylamide/sodium acryloyldimethyl taurate copolymer/isohexadecane/polysorbate 80, ammonium acryloyldimethyltaurate/VP copolymer, Sodium Acrylate/Sodium Acryloyldimethyl Taurate Copolymer, acrylates copolymer, Acrylates Crosspolymer-4, Acrylates Crosspolymer-3, acrylates/beheneth-25 methacrylate copolymer, acrylates/C10-C30 alkyl acrylate crosspolymer, acrylates/steareth-20 itaconate copolymer, ammonium polyacrylate/Isohexadecane/PEG-40 castor oil; carbomer, sodium carbomer, crosslinked polyvinylpyrrolidone (PVP), polyacrylamide/C13-14 isoparaffin/laureth-7, polyacrylate 13/polyisobutene/polysorbate 20, polyacrylate crosspolymer-6, polyamide-3, polyquaternium-37 (and) hydrogenated polydecene (and) trideceth-6, Acrylamide/Sodium Acryloyldimethyltaurate/Acrylic Acid Copolymer, sodium acrylate/acryloyldimethyltaurate/dimethylacrylamide, crosspolymer (and) isohexadecane (and) polysorbate 60, sodium polyacrylate.

8. A composition according to claim 1 wherein the composition has a selenium sulfide cake 90 mm penetration depth peak force of less than 200 grams after 24 hours at 40 C.

9. A composition according to claim 1 wherein the composition has a cake height of greater than 30 mm after 24 hours at 40 C.

10. A composition according to claim 1 further comprising an oxidizing agent.

11. A composition according to claim 10 wherein the oxidizing agent is from about 0.1% to about 0.4% of an oxidizing agent.

12. A composition according to claim 10 wherein the oxidizing agent is selected from the group consisting of hydrogen peroxide, sulfur, colloidal sulfur and mixtures thereof.

13. A composition according to claim 1 further comprising a preservative.

14. A composition according to claim 1 wherein the preservative is selected from the group consisting of sodium salicylate, sodium benzoate, phenoxyethanol, Methylchloroisothiazolinone (CMIT) and Methylisothiazolinone (MIT) and mixtures thereof.

15. A composition according to claim 14 wherein the preservative is from about 0.1% to about 1.0%.

16. A composition according to claim 1 further comprising from about 38% to about 58% of a liquid carrier.
